(19) United States
(12) Patent Application Publication
(10) Pub. No.: US 2025/0111082 A1
(43) Pub. Date: Apr. 3, 2025

(54) Secure Heartbeat for Sensor Indicator

(71) Applicant: Apple Inc., Cupertino, CA (US)

(72) Inventors: Peter A Lisherness, Los Gatos, CA (US); Assaf Menachem, Hod Hasharon (IL)

(21) Appl. No.: 18/895,200

(22) Filed: Sep. 24, 2024

Related U.S. Application Data
(60) Provisional application No. 63/541,215, filed on Sep. 28, 2023.

(51) Int. Cl. G06F 21/62; A61B 5/00; A61B 5/024

(52) U.S. Cl. CPC G06F 21/6245; A61B 5/02438; A61B 5/743

(57) ABSTRACT

Systems and methods for providing secure access to a sensor of an electronic device is provide. Such a system may include an electronic device with an electronic display to display an indicator associated with a sensor. The electronic device may use image processing circuitry to process image data corresponding to the indicator. The sensor may correspond to the indicator and may be controlled according to secure software running in a trusted execution environment. The secure software may enable or disable access to the sensor after a defined period of time in response to receiving, or not receiving, a message indicating that the indicator is being displayed on the electronic display.

### CROSS-REFERENCE TO RELATED APPLICATIONS

This application claims priority to U.S. Application No. 63/541,215, filed Sep. 28, 2023, entitled “Secure Heartbeat for Sensor Indicator,” which is incorporated by reference herein in its entirety for all purposes.

### BACKGROUND

This disclosure relates to image processing circuitry to ensure that a sensor indicator is inserted into image content when a sensor of a device is active or recently active even in the event of a software malfunction.

People desire to trust that the electronic sensors of their electronic devices will not be used without their permission or awareness. As such, some electronic devices display special indicators in image content to indicate visually when a sensor, such as a camera or microphone, is currently or was recently in use. That is, the displayed frame may include secure content for privacy-oriented purposes, such as a camera indication light (CIL) or a microphone indication light (MIL). This allows the person using the device to be aware when those sensors have been activated. As such, the secure content must be guaranteed to be presented to the user as intended. However, since these indicators are part of the image content, it is possible that a malfunction in the software that generates image content could result in the indicators not appearing in the image content, even if the sensors are activated.

### SUMMARY

Embodiments herein are directed to exclave architecture for image processing circuitry that may provide a “secure heartbeat” signal to sensor hardware to verify that a sensor indicator is appearing on the electronic display. As long as the sensor hardware has received a secure heartbeat signal from the image processing circuitry within a threshold amount of time, the sensor hardware may allow access to the sensor data from the sensor hardware. When the sensor hardware has not received a secure heartbeat signal for more than the threshold amount of time, however, the sensor hardware may stop providing access to the sensor data. This may prevent sensitive personal data from the sensor hardware from being accessed without the user's knowledge. This is achieved using secure exclave architecture, or other trusted software, executed in a trusted execution environment of the display pipeline configured to render frames for presentation on a display of device. Such “secure heartbeat” software and/or exclave architecture may work together with sensor software and/or hardware to disable the flow of sensor data via a dead-man switch, for example, whenever the sensor data is available outside the exclave yet no sensor indicator is visible. As such, the visibility of a sensor indicator may be regularly or periodically reported to the sensor software and/or hardware, thereby generating a secure “heartbeat” or message to serve as a temporary permission to expose the sensor to untrusted software.

### BRIEF DESCRIPTION OF THE DRAWINGS

Various aspects of this disclosure may be better understood upon reading the following detailed description and upon reference to the drawings in which:

FIG. 1 is a schematic diagram of an electronic device that includes sensors and an electronic display, in accordance with an embodiment;

FIG. 2 is an example of the electronic device of FIG. 1 in the form of a handheld device, in accordance with an embodiment;

FIG. 3 is another example of the electronic device of FIG. 1 in the form of a tablet device, in accordance with an embodiment;

FIG. 4 is another example of the electronic device of FIG. 1 in the form of a computer, in accordance with an embodiment;

FIG. 5 is another example of the electronic device of FIG. 1 in the form of a watch, in accordance with an embodiment;

FIG. 6 is another example of the electronic device of FIG. 1 in the form of a computer, in accordance with an embodiment;

FIG. 7 is a schematic diagram of a sensor indicator being displayed on an electronic device when certain sensors are active, in accordance with an embodiment;

FIG. 8 is a block diagram demonstrating sharing data with non-exclave software using an audio sensor and a display indicator, in accordance with an embodiment;

FIG. 9 is a block diagram of the image processing circuitry and the trusted and non-trusted aspects of the image processing circuitry, in accordance with an embodiment; and

FIG. 10 is a flowchart of an example process for generating the secure heartbeat indicator, in accordance with an embodiment.

### DETAILED DESCRIPTION

One or more specific embodiments of the present disclosure will be described below. These described embodiments are only examples of the presently disclosed techniques. Additionally, in an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but may nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Furthermore, the phrase A “based on” B is intended to mean that A is at least partially based on B. Moreover, the term “or” is intended to be inclusive (e.g., logical OR) and not exclusive (e.g., logical XOR). In other words, the phrase A “or” B is intended to mean A, B, or both A and B.

Electronic devices may use specialized hardware sometimes referred to as “enclaves” that may use physical separation techniques to create a secure environment and prevent external entities (e.g., executing processes, the CPU, etc.) from being able to directly access internal data. A secure environment may be confined to a particular region of a device that can be controlled and does not extend to other regions outside of that control. However, a secure environment may be extended by implementing secure exclaves. As described in more detail below, a computing device may include one or more processors to co-execute trusted processes and untrusted processes in an isolated manner such that the trusted processes may provide checks on the untrusted processes.

Electronic devices often use electronic displays to present visual information, including a special indicator to visually indicate when a sensor of the electronic device is or was recently in use. Such electronic devices may include any device with a display and one or more sensors, such as a microphone or camera. For example, such devices may include computers, mobile phones, portable media devices, tablets, televisions, virtual-reality headsets, and vehicle dashboards, among many others. Visual indicators that signal to a user of the electronic device that a sensor of the device is active may serve as a security measure in that the user may be made aware of when the electronic device is collecting visual or audio data from the user via a microphone or camera, for instance. The image processing circuitry of the electronic device may include exclave architecture that ensures the sensor indicators are always inserted into image content of the display, even if there is a software malfunction, thereby improving the security of the electronic device. Such secure exclave architecture may be implemented with or without additional secure circuitry, such as a secure content layer in the image processing circuitry, designated for providing a secure content indicator, as described in more detail below.

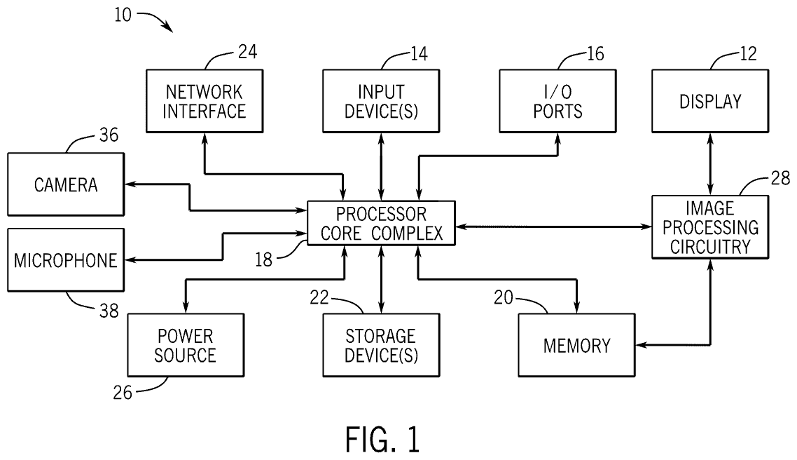

With the foregoing in mind, FIG. 1 is an example electronic device 10 that may disable sensor data when an indicator is not displayed on an electronic display 12 of the electronic device 10. As described in more detail below, the electronic device 10 may be any suitable electronic device, such as a computer, a mobile phone, a portable media device, a tablet, a television, a virtual-reality headset, a wearable device such as a watch, a vehicle dashboard, or the like. Thus, it should be noted that FIG. 1 is merely one example of a particular implementation and is intended to illustrate the types of components that may be present in an electronic device 10.

The electronic device 10 may include one or more electronic displays 12, input devices 14, input/output (I/O) ports 16, a processor core complex 18 having one or more processors or processor cores, local memory 20, a main memory storage device 22, a network interface 24, a power source 26, image processing circuitry 28, a camera 36, and a microphone 38. The various components described in FIG. 1 may include hardware elements (e.g., circuitry), software elements (e.g., a tangible, non-transitory computer-readable medium storing instructions), or a combination of both hardware and software elements. As should be appreciated, the various components may be combined into fewer components or separated into additional components. For example, the local memory 20 and the main memory storage device 22 may be included in a single component. Moreover, the image processing circuitry 28 (e.g., a graphics processing unit, a display image processing pipeline, etc.) may be included in the processor core complex 18 or be implemented separately.

The processor core complex 18 is operably coupled with local memory 20 and the main memory storage device 22. Thus, the processor core complex 18 may execute instructions stored in local memory 20 or the main memory storage device 22 to perform operations, such as generating or transmitting image data to display on the electronic display 12. As such, the processor core complex 18 may include one or more general purpose microprocessors, one or more application specific integrated circuits (ASICs), one or more field programmable logic arrays (FPGAs), or any combination thereof.

In addition to program instructions, the local memory 20 or the main memory storage device 22 may store data to be processed by the processor core complex 18. Thus, the local memory 20 and/or the main memory storage device 22 may include one or more tangible, non-transitory, computer-readable media. For example, the local memory 20 may include random access memory (RAM) and the main memory storage device 22 may include read-only memory (ROM), rewritable non-volatile memory such as flash memory, hard drives, optical discs, or the like.

The network interface 24 may communicate data with another electronic device or a network. For example, the network interface 24 (e.g., a radio frequency system) may enable the electronic device 10 to communicatively couple to a personal area network (PAN), such as a BLUETOOTH® network, a local area network (LAN), such as an 802.11x Wi-Fi network, or a wide area network (WAN), such as a 4G, Long-Term Evolution (LTE), or 5G cellular network.

The power source 26 may provide electrical power to operate the processor core complex 18 and/or other components in the electronic device 10. Thus, the power source 26 may include any suitable source of energy, such as a rechargeable lithium polymer (Li-poly) battery and/or an alternating current (AC) power converter.

The I/O ports 16 may enable the electronic device 10 to interface with various other electronic devices. The input devices 14 may enable a user to interact with the electronic device 10. For example, the input devices 14 may include buttons, keyboards, mice, trackpads, and the like. Additionally or alternatively, the electronic display 12 may include touch sensing components that enable user inputs to the electronic device 10 by detecting occurrence and/or position of an object touching its screen (e.g., surface of the electronic display 12).

The electronic display 12 may display a graphical user interface (GUI) (e.g., of an operating system or computer program), an application interface, text, a still image, and/or video content. The electronic display 12 may include a display panel with one or more display pixels to facilitate displaying images. Additionally, each display pixel may represent one of the sub-pixels that control the luminance of a color component (e.g., red, green, or blue). As used herein, each display pixel corresponds to one sub-pixel (e.g., a red, green, or blue subpixel).

As described above, the electronic display 12 may display an image by controlling the luminance output (e.g., light emission) of the sub-pixels based on corresponding image data. In some embodiments, pixel or image data may be generated by or received from an image source, such as the processor core complex 18, a graphics processing unit (GPU), storage device 22, or an image sensor (e.g., camera). Additionally, in some embodiments, image data may be received from another electronic device 10, for example, via the network interface 24 and/or an I/O port 16. Moreover, in some embodiments, the electronic device 10 may include multiple electronic displays 12 and/or may perform image processing (e.g., via the image processing circuitry 28) for one or more external electronic displays 12, such as connected via the network interface 24 and/or the I/O ports 16.

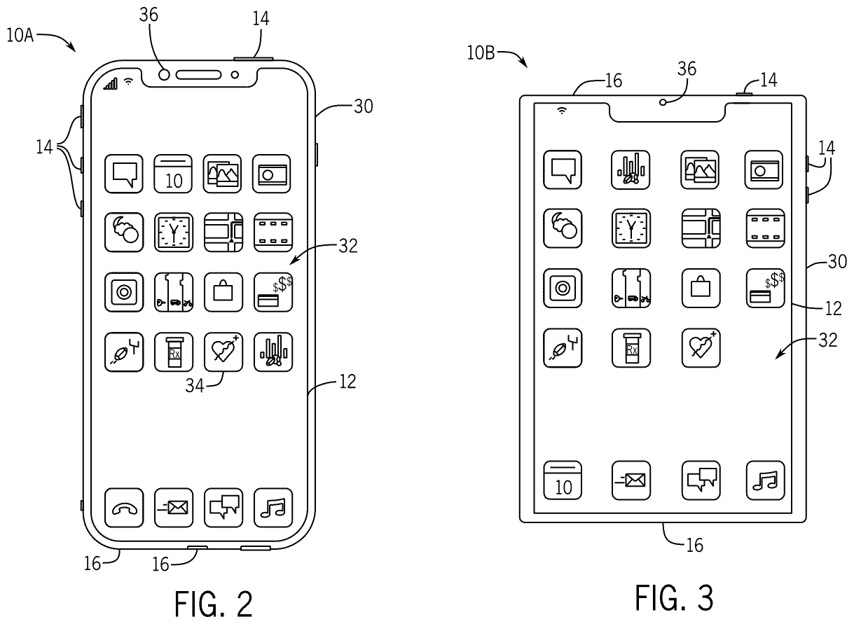

The electronic device 10 may be any suitable electronic device. To help illustrate, one example of a suitable electronic device 10, specifically a handheld device 10A, is shown in FIG. 2. In some embodiments, the handheld device 10A may be a portable phone, a media player, a personal data organizer, a handheld game platform, and/or the like. For illustrative purposes, the handheld device 10A may be a smartphone, such as an IPHONE® model available from Apple Inc.

The handheld device 10A may include an enclosure 30 (e.g., housing) to, for example, protect interior components from physical damage and/or shield them from electromagnetic interference. The enclosure 30 may surround, at least partially, the electronic display 12. In the depicted embodiment, the electronic display 12 is displaying a graphical user interface (GUI) 32 having an array of icons. By way of example, when an icon is selected either by an input device 14 or a touch-sensing component of the electronic display 12, an application program may launch.

Input devices 14 may be accessed through openings in the enclosure 30. Moreover, the input devices 14 may enable a user to interact with the handheld device 10A. For example, the input devices 14 may enable the user to activate or deactivate the handheld device 10A, navigate a user interface to a home screen, navigate a user interface to a user-configurable application screen, activate a voice-recognition feature, provide volume control, and/or toggle between vibrate and ring modes. Moreover, the I/O ports 16 may also open through the enclosure 30. Additionally, the electronic device may include one or more cameras 36 to capture pictures or video. In some embodiments, a camera 36 may be used in conjunction with a virtual reality or augmented reality visualization on the electronic display 12.

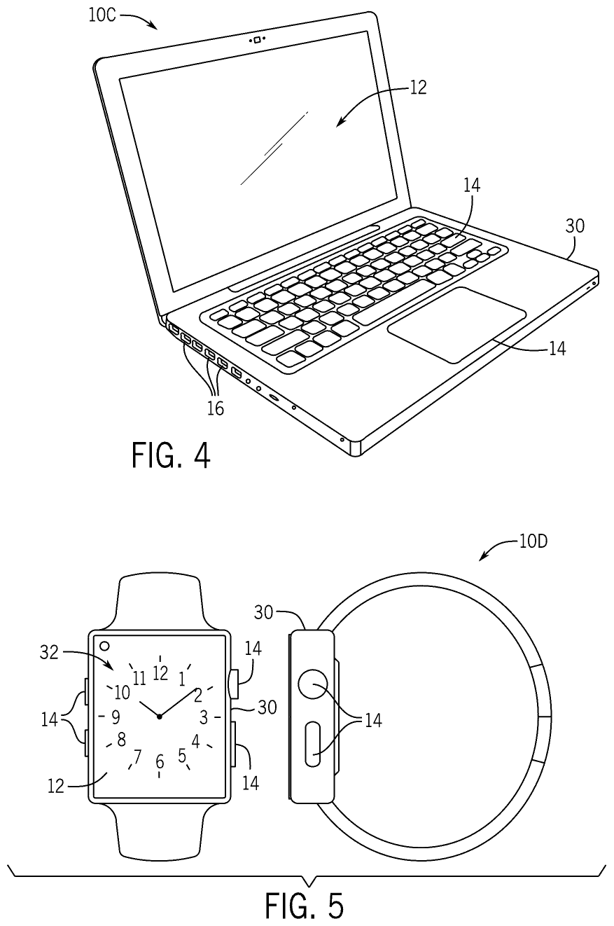

Another example of a suitable electronic device 10, specifically a tablet device 10B, is shown in FIG. 3. For illustration purposes, the tablet device 10B may be an IPAD® model available from Apple Inc. A further example of a suitable electronic device 10, specifically a computer 10C, is shown in FIG. 4. For illustrative purposes, the computer 10C may be a MACBOOK® or IMAC® model available from Apple Inc. Another example of a suitable electronic device 10, specifically a watch 10D, is shown in FIG. 5. For illustrative purposes, the watch 10D may be an APPLE WATCH® model available from Apple Inc. As depicted, the tablet device 10B, the computer 10C, and the watch 10D each also includes an electronic display 12, input devices 14, I/O ports 16, and an enclosure 30. The electronic display 12 may display a GUI 32. Here, the GUI 32 shows a visualization of a clock. When the visualization is selected either by the input device 14 or a touch-sensing component of the electronic display 12, an application program may launch, such as to transition the GUI 32 to presenting the icons discussed in FIGS. 2 and 3.

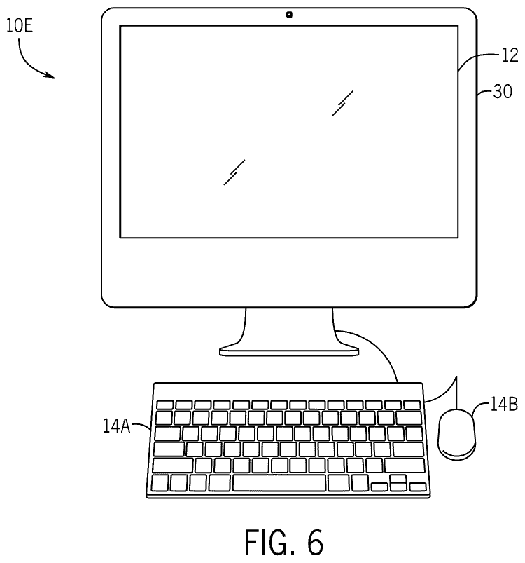

Turning to FIG. 6, a computer 10E may represent another embodiment of the electronic device 10 of FIG. 1. The computer 10E may be any suitable computer, such as a desktop computer, a server, or a notebook computer, but may also be a standalone media player or video gaming machine. By way of example, the computer 10E may be an IMAC®, a MACBOOK®, or other similar device by Apple Inc. of Cupertino, California. It should be noted that the computer 10E may also represent a personal computer (PC) by another manufacturer. A similar enclosure 30 may be provided to protect and enclose internal components of the computer 10E, such as the electronic display 12. In certain embodiments, a user of the computer 10E may interact with the computer 10E using various peripheral input devices 14, such as a keyboard 14A or mouse 14B, which may connect to the computer 10E.

As described above, the electronic display 12 may display images based at least in part on image data. Before being used to display a corresponding image on the electronic display 12, the image data may be processed, for example, via image processing circuitry, as described in further detail below.

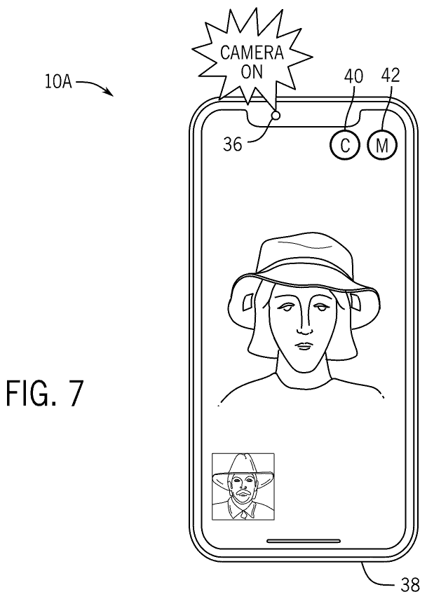

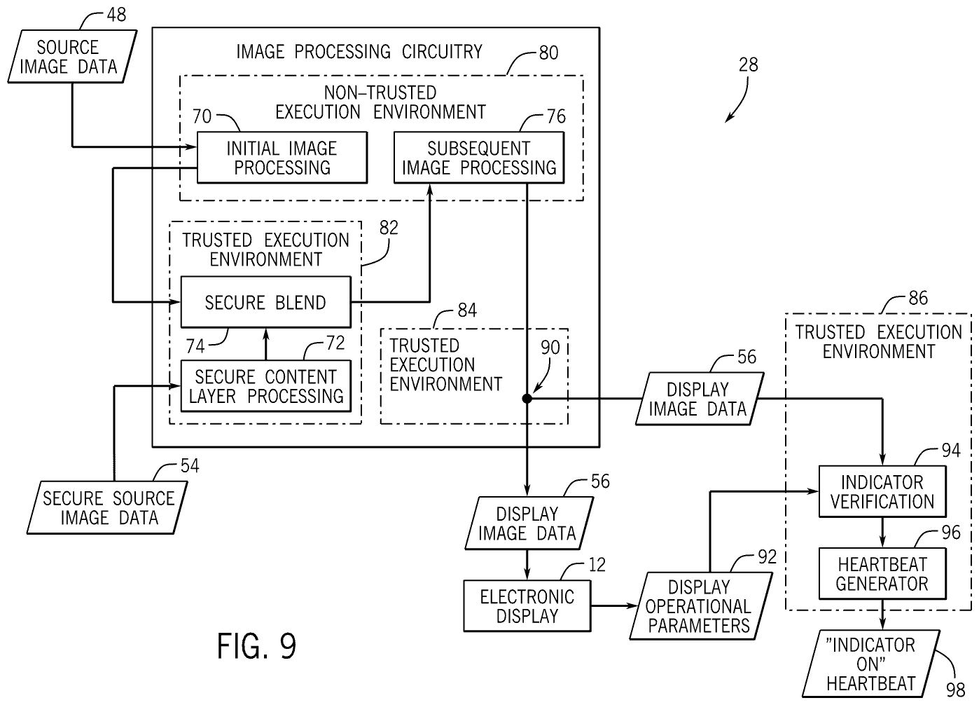

To help illustrate how a sensor indicator may be displayed on the electronic display when certain sensors (e.g., camera, microphone) are on, FIG. 7 shows, for example, a handheld device 10A with a display 12 and an audio sensor or transducer (e.g. a microphone) 38. As shown in FIG. 7, the handheld device 10A may be used to conduct a videoconference in which the camera 36 and the microphone 38 are activated and in use. In that case, the sensor indicator may be displayed on the electronic display 12 of the handheld device 10A to indicate to the user of the device that certain sensors are in use. For example, a camera indication light (CIL), which is an indicator associated with the camera 40, and/or a microphone indicator light (MIL), which is an indicator associated with the audio sensor or microphone 42, may be displayed at the top of the screen in response to the camera 36 and the audio sensor 38 being used. That is, the sensor indicators 40, 42 may be inserted or triggered when one or more sensors and/or applications of a device are activated or otherwise communicate with image processing circuitry, as described in more detail below. In this way, both the sensor indicator and the display content, which may be content associated with untrusted software (e.g. the videoconference), are both displayed on the electronic display 12 simultaneously. It should be noted that the sensor indicator may be a secure content indicator that is inserted into the display image data via secure image processing circuitry, as described in more detail below with respect to FIG. 9.

In FIG. 7, the sensor indicators 40, 42 are shown to appear as a dot on the electronic display 12; however, it should be noted that the indicator may be any suitable type of indicator and, therefore, may appear in a variety of colors or shapes, including text. For example, the sensor indicator associated with the camera 40 may include the words “CAMERA ON”, just the letter “C”, a colored dot, the outline of a dot, a star, an icon of a camera, or any combination thereof. Further, the indicator may appear in a fixed position on the electronic display 12 or it may move around the display or be animated. Additionally, although FIG. 7 features an example of the indicators 40, 42 being applied in a videoconference, it should be noted that the indicators 40, 42 may be applied in any instance in which a sensor of a device 10 is in use, and therefore collecting private or secure content such as audio or visual data and sharing that data with untrusted software (e.g. a third-party application). As such, the indicators 40, 42 may be applied to any private or secure function or operation of the device that a user may want to be alerted about when such functions and associated sensors are active and/or in use. For example, a user may want sensor indicators applied when using gyroscope sensors, WiFi, Face ID or other forms of facial recognition, digital payment or personal finance applications, location-tracking or navigation applications, or other sensors or applications that may track and/or share private or secure data collected via one or more device sensors.

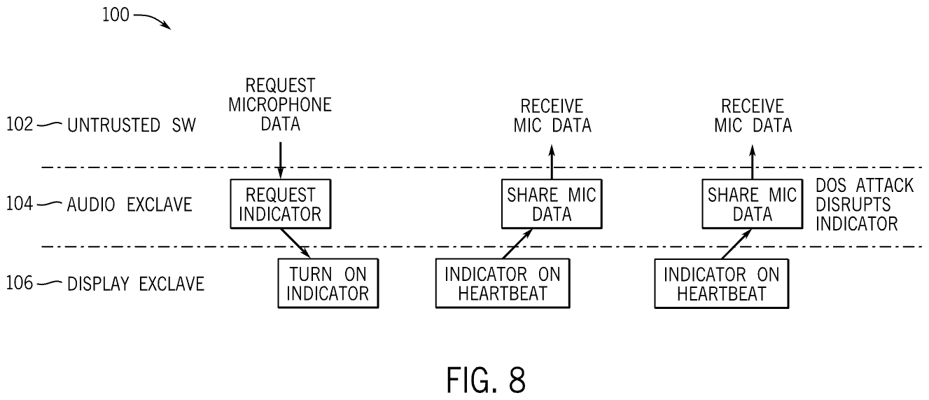

As mentioned above, display of the sensor indicator while a sensor is or was recently in use may be guaranteed in order to provide the intended security benefit of the indicator, which may be achieved via the image processing circuitry of the device. However, sensor exclave scheduling may remain the domain of untrusted software. As such, the security of the system may therefore be robust to denial of service attacks. For the display, this means that the visibility of a sensor indicator may be regularly reported to the sensor exclave software and/or hardware, thereby generating a secure heartbeat, or secure message, as a fail-secure temporary permission to expose the sensor data to untrusted software. This secure heartbeat process 100 is demonstrated in FIG. 8. As shown in FIG. 8, an untrusted software 102, such as a third-party application, may request sensor data, such as microphone data. In response to this request for microphone data, the audio exclave 104 may request monitoring software in the display exclave 106 to provide a microphone sensor indicator to appear on the display. In turn, the display exclave 106 may turn on the microphone sensor indicator and provide a heartbeat message to the audio exclave 104 that the microphone indicator is visible. In response to periodically receiving the heartbeat message that the microphone indicator is visible, the audio exclave 104 may share the microphone data with the untrusted software 102. However, as soon as the indicator is disrupted for any reason and the monitoring software of the display exclave 106 stops sending an “indicator on” heartbeat to the audio exclave 104 for some threshold period of time, the audio exclave 104 will stop making microphone data available to the untrusted software 102.

It should be noted that the mechanism of sharing data with non-exclave, untrusted software is expected to differ depending on the sensor being used or activated. For example, microphone data may enter the system under exclave control and may be released when the microphone indicator is triggered by an exclave process. That is, no hardware may be directly involved in the release of the microphone data. As such, enforcing the policy in the presence or absence of the microphone indicator heartbeat may be entirely a matter of software. Camera data, on the other hand, may have higher bandwidth requirements than other sensors in order to be moved around by software. As such, special designated hardware may be involved in providing the camera data to the camera exclave. Such designated hardware may be able to receive sensor data irrespective of the indicator state and store it in exclave-controlled memory, or DRAM. A dead-man switch may be implemented on the remaining sensor data paths that may block the flow of pixel data associated with the camera if the switch does not regularly receive a heartbeat indicating that the camera indicator is on.

With the foregoing in mind, FIG. 9 demonstrates the image processing circuitry involved in generating the heartbeat signal to the sensor software and/or hardware. As mentioned above, in some embodiments, the secure exclave architecture or trusted software responsible for generating the heartbeat may be implemented with or without additional secure circuitry, such as a secure content layer in the image processing circuitry. For example, a secure content layer may be implemented in the image processing circuitry for providing a secure sensor indicator in addition to implementing the heartbeat software to ensure the secure indicator is always displayed, as shown in FIG. 9. In such embodiments, the sensor indicator may be treated as secure image data generated by the secure content layer and blended in a trusted execution environment so as to prevent tampering with the sensor indicator itself prior to display. Although FIG. 9 represents an embodiment that includes a secure content layer in the image processing circuitry, it should be noted that the heartbeat software may be implemented with or without such additional secure circuitry.

Continuing with FIG. 9, in accordance with an embodiment, the image processing circuitry 28 may process image data, including non-secure image data and secure image data. In the present embodiment, the non-secure image data may include image data other than the sensor indicator, or the secure content indicator, while the secure image data may include the sensor indicator (e.g., the secure content indicator). Both the non-secure image data and the secure image data may be processed for display on one or more electronic displays 12. The image processing circuitry 28 may be implemented in the electronic device 10, in the electronic display 12, or a combination thereof. For example, the image processing circuitry 28 may be included in the processor core complex 18, a timing controller (TCON) in the electronic display 12, or any combination thereof. As should be appreciated, although image processing is discussed herein as being performed via a number of image data processing blocks, embodiments may include hardware and/or software components to carry out the techniques discussed herein. For example, the image processing circuitry 28, which may include both trusted and non-trusted execution environments, may include a display pipeline and additional hardware or software to process image data. That is, the display pipeline may include circuitry to process input data to produce output frames. As such, the display pipeline may perform various operations such as panel compensation, swizzling, dithering, cropping, timing control, DisplayPort™ transmission, etc. In this way, image data may be processed via the display pipeline of the image processing circuitry 28 to reduce or eliminate image artifacts, compensate for one or more different software or hardware related effects, and/or format the image data for display on one or more electronic displays 12. With respect to displaying the indicator, the display pipeline may include a secure blend unit, a secure extractor, a secure direct memory access (DMA) engine securely fetch and blend the indicator into the image data to be displayed on the electronic display. In other embodiments, the DMA engine may not be considered as part of the display pipeline.

The image processing circuitry 28 may receive source image data 48 corresponding to a desired image to be displayed on the electronic display 12. The source image data 48 may include non-secure image data, or any image data to be displayed that does not include the secure content indicator. The source image data 48 may indicate target characteristics (e.g., pixel data) corresponding to the desired image using any suitable source format, such as an RGB format, an αRGB format, a YCbCr format, and/or the like. Moreover, the source image data 48 may be fixed or floating point and be of any suitable bit-depth. Furthermore, the source image data 48 may reside in a linear color space, a gamma-corrected color space, or any other suitable color space. Additionally, the image processing circuitry 28 may receive secure source image data 54 corresponding to a secure content indicator (e.g., the sensor indicator) to be displayed on the electronic display 12. Like the source image data 48, the secure source image data 54 may indicate target characteristics (e.g., pixel data) corresponding to the desired image (e.g. secure content indicator) using any suitable source format, such as an RGB format, an αRGB format, a YCbCr format, and/or the like. Moreover, like the source image data 48, the secure source image data 54 may be a fixed or floating point and be of any suitable bit-depth. Furthermore, the secure source image data 54 may reside in a linear color space, a gamma-corrected color space, or any other suitable color space. Moreover, as used herein, pixel data/values of image data may refer to individual color component (e.g., red, green, and blue) data values corresponding to pixel positions of the display panel.

The image processing circuitry 28 may operate to process source image data 48 and secure source image data 54. As previously discussed, the source image data 48 may include any and all image data to be displayed that is not the secure source image data 54 (e.g., the secure content indicator). As such, the source image data 48 may include captured images (e.g., from one or more cameras 36), images stored in memory, graphics generated by the processor core complex 18, or a combination thereof. The secure source image data 54 may include images stored in memory. For example, the sensor indicator may include image data fetched from dedicated DMA of the secure content layer.

As previously mentioned, the functions and operations performed by the image processing circuitry 28 may be divided between various image data processing blocks (e.g., circuitry, modules, or processing stages) that are part of the display pipeline described above. The term “block”, as used herein, may or may not mean a logical or physical separation between the image data processing blocks. Such image data processing blocks may include one or more burn-in compensation (BIC)/burn-in statistics (BIS) blocks, a pixel contrast control (PCC) block, color management block, a dither block, a blend block, a warp block, a scaling/rotation block, etc. Such image data processing blocks may receive and process the source image data 48 and output display image data 56 in a format (e.g., digital format, image space, and/or resolution) interpretable by the electronic display 12. Simultaneously, but separately, the image processing circuitry 28 may also receive and process the secure source image data 54 and output the secure content indicator in a format interpretable by the electronic display 12, as further described more detail below. After processing, the image processing circuitry 28 may output the display image data 56, which may include both the secure content indicator and the non-secure image data, to the electronic display 12.

Continuing with FIG. 9, FIG. 9 illustrates an overall arrangement of the secure and non-secure aspects of the image processing circuitry 28. As discussed above, the source image data 48, which may include non-secure image data, or any image data to be displayed that does not include the secure content indicator may undergo initial image processing 70 via various processing blocks of the display pipeline located in a non-trusted execution environment 80 of the image processing circuitry 28. The initial image processing 70 may occur in software, hardware, or both, whereas the secure content layer processing 72 may occur in hardware to reduce attack surface. It should be noted that only vetted software, such as the exclave architecture responsible for generating the secure heartbeat, may have access to a hardware trusted execution environment 84, 86. Simultaneous with the initial image processing 70 of the source image data 48, the secure source image data 54 may be fetched by dedicated DMA to undergo secure content layer processing 72 in the hardware trusted execution environment 82. It should be noted that only vetted software may have access to the DMA. The secure source image data 54 may then be blended via the secure blend block 74. It should be noted that the secure blend block 74 is located in the trusted execution environment 82. Blending may be performed in a linear space and may include one or more blending modes that are independently configurable.

Also blended at the secure blend block 74 is the source image data 48 after initial image processing 70. That is, the source image data 48 and the secure source image data 54 are both blended in the secure blend block 74. As such, the secure blend 74 includes both the secure content indicator generated via the secure source image data 54 as well as the non-secure image data generated via the source image data 48. The blended secure and non-secure data are then sent for subsequent image processing 76 in the rich, or non-trusted, execution environment 80. The blended image may be sent to the output port 90 in the trusted execution environment 84 prior to being sent out as display image data 56. The display image data 56, which contains the blended secure and non-secure image data, may then be displayed via the electronic display 12. Additionally, in some embodiments, as shown in FIG. 9, the electronic display 12 may provide one or more display operational parameters 92 and confirm that such parameters are provided. The one or more operational parameters may include confirming the display is on, that the brightness is at a certain level, or other operational parameters related to the display of the display image data 56 and/or the user's ability to see the indicator. In this way, the display may provide the appropriate conditions for ensuring that the display image data 56 (e.g., the indicator) is always in view.

As previously mentioned, trusted software executed in a trusted execution environment 86 is responsible for generating the heartbeat to the sensor that is providing the sensor data. For example, when the trusted heartbeat software verifies that the indicator is on-screen via indicator verification 94, verification is sent to the heartbeat generator 96 and an “indicator on” heartbeat 98 may be generated to the sensor. In this way, the trusted monitoring software may know the state of the secure indicatory layer responsible for compositing the indicators onto the image. It should be noted that the heartbeat software may only begin sending heartbeats if it is notified by the indicator verification software via indicator verification 94 that the layer is enabled. Similarly, the indicator verification software may only disable the secure indicatory layer, or the sensor indicator, after it has notified the heartbeat software that the indicator is turned off. In this way, a potential attacker is prevented from denying communication between the heartbeat software and the indicator control of the sensor.

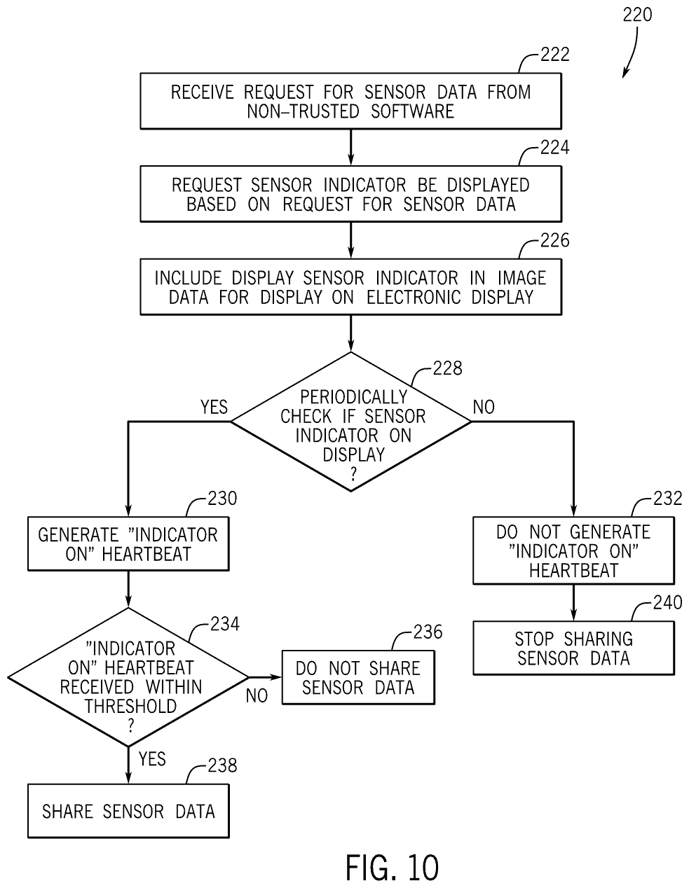

Referring now to FIG. 10, FIG. 10 is a flowchart 220 of an example process for generating the secure heartbeat indicator. At process block 222, the exclave architecture of the sensor may receive a request for sensor data from an untrusted software. The untrusted software may be a third-party application. For example, a third-party videoconference application may request the camera 36 to share camera data with the application. At process block 224, the sensor exclave architecture may then request a sensor indicator be displayed based on the request for the sensor data. For example, the camera exclave, in response to receiving a request to share camera data with the videoconference application, may request the image processing circuitry 28 to display the camera sensor indicator on the display image. At process block 226, the sensor indicator may be displayed on the electronic display to signal to the user of the device that the camera sensor is on and that camera data is being shared with untrusted software. At process block 228, the heartbeat software may periodically check the state of the display for whether the sensor indicator is displayed. The frequency at which the heartbeat software checks the display may vary depending on the software and the security policy of the software. For example, the heartbeat software may check the display every few seconds to every few milliseconds.

At process block 232, if the sensor indicator is not displayed, the heartbeat software may not generate a heartbeat or message. At process block 240, in response to an absence of the heartbeat, the sensor exclave may stop sharing sensor data. If, however, the sensor indicator is displayed, at process block 230, the heartbeat software may generate an “indicator on” heartbeat, or “indicator on” message. In this way, the heartbeat software may be said to periodically generate the heartbeat message based on the periodic checks for the sensor indicator. In some embodiments, as depicted in process block 234 of FIG. 10, the sensor exclave may determine whether the heartbeat was received within a threshold amount of time. The threshold amount of time may vary depending on the software policy. That is, sensor data may only be shared for a defined period of time, and the amount of time sensor data is allowed to be shared after each heartbeat is received is a matter of software policy. For example, the defined period of time may be 1 second, 2 seconds, 3 seconds, 4 seconds, or 5 seconds. In another example, the defined period of time may be some amount of time less than 1 second but greater than 1 millisecond. If the heartbeat is received within the threshold amount of time, at process block 238, the sensor exclave will share the sensor data with the untrusted software. If the heartbeat is not received within the threshold amount of time, however, at process block 236, the sensor exclave will not share the sensor data. In this way, the sensor exclave and the heartbeat software of the image processing circuitry may work together to enable the flow of sensor data when the heartbeat message is received and disable the flow of sensor data when the heartbeat message is not received, thereby providing security guarantees with the use of untrusted software.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

It is well understood that the use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform] ing [a function] . . . ” or “step for [perform] ing [a function] . . . ”, it is intended that such elements are to be interpreted under 35 U.S.C. 112 (f). However, for any claims containing elements designated in any other manner, it is intended that such elements are not to be interpreted under 35 U.S.C. 112 (f).

What is claimed is:

1. A method comprising:
receiving a request to access a sensor of an electronic device;
based on the request to access the sensor, displaying an indicator on an electronic display of the electronic device indicating that the sensor is in use;
periodically generating a message indicating that the indicator is being displayed;
enabling access to the sensor for a defined period of time upon receipt of the message indicating that the indicator is being displayed; and
disabling access to the sensor after the defined period of time if a subsequent message indicating that the indicator is being displayed is not received.

2. The method of claim 1, wherein the request to access the sensor is received into sensor exclave software running in a trusted execution environment, and wherein the request is from second software running outside of the trusted execution environment.

3. The method of claim 2, wherein displaying the indicator is controlled by display exclave software controlling image processing circuitry in the trusted execution environment.

4. The method of claim 2, wherein the second software comprises untrusted software not running in the trusted execution environment.

5. The method of claim 4, wherein the untrusted software comprises a third-party application.

6. The method of claim 1, wherein the message comprises a heartbeat message occurring at a regular cadence, and wherein the subsequent message comprises a subsequent heartbeat message occurring at the regular cadence.

7. The method of claim 1, wherein the defined period of time is less than 5 seconds and greater than 1 millisecond.

8. An electronic device comprising:
an electronic display comprising display pixels configured to display an image and one or more indicators associated with one or more sensors;
image processing circuitry communicatively coupled to the electronic display and configured to process image data corresponding to the image and the one or more indicators; and
the one or more sensors, wherein the one or more sensors correspond to the one or more indicators and are controlled according to sensor exclave software running in a trusted execution environment, wherein the sensor exclave software is configured to:
enable access to the one or more sensors for a defined period of time in response to receiving a message indicating that the one or more indicators are being displayed; and
disable access to the one or more sensors after the defined period of time if a subsequent message indicating that the one or more indicators are being displayed is not received.

9. The electronic device of claim 8, wherein the message comprises a heartbeat message occurring at a regular cadence, and wherein the subsequent message comprises a subsequent heartbeat message occurring at the regular cadence.

10. The electronic device of claim 9, wherein the heartbeat message and the subsequent heartbeat message are generated by monitoring software configured to run in the trusted execution environment controlling the image processing circuitry.

11. The electronic device of claim 8, wherein the one or more indicators being displayed are indicative of the one or more sensors being used.

12. The electronic device of claim 8, wherein the defined period of time is less than 5 seconds and greater than 1 millisecond.

13. The electronic device of claim 8, wherein the sensor exclave software is configured to run in the trusted execution environment.

14. A method for ensuring display of a sensor indicator on a user device, comprising:
receiving, via sensor exclave software running in a trusted execution environment, a request to access a sensor of an electronic device;
based on the request to access the sensor, requesting, via the sensor exclave software, that the sensor indicator be displayed;
including, via display exclave software, the sensor indicator in image data for display on an electronic display of the user device;
periodically checking, via the display exclave software, if the sensor indicator is being displayed;
in response to the sensor indicator being displayed, generating, via the display exclave software, a message to the sensor exclave software that the sensor indicator is being displayed; and
in response to receiving the message, providing access, via the sensor exclave software, to sensor data.

15. The method of claim 14, wherein the request is from non-trusted software.

16. The method of claim 14, wherein the request to display the sensor indicator is sent to the display exclave software, and wherein the display exclave software controls image processing circuitry of the user device.

17. The method of claim 14, comprising, in response to not receiving the message, preventing access, via the sensor exclave software, to the sensor data.

18. The method of claim 14, wherein the message comprises a heartbeat message.

19. The method of claim 14, comprising discontinuing access, via the sensor exclave software, to the sensor data after a defined period of time if a subsequent message is not received.

20. The method of claim 19, wherein the defined period of time is less than 5 seconds and greater than 1 millisecond.
